SEQUENCE LISTING (3 sequences)

SEQ ID NO: 1 - protein, 406 aa, Artificial
  Synthetic
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - protein, 7 aa, Artificial
  Synthetic
KLAKLAK

SEQ ID NO: 3 - protein, 14 aa, Artificial
  Synthetic
KLAKLAKKLAKLAK

(19) United States
(12) Patent Application Publication
(10) Pub. No.: US 2004/0072755 A1
(43) Pub. Date: Apr. 15, 2004

(54) TF antagonist

(76) Inventors: Henning Ralf Stennicke, Kokkedal (DK); Soren E. Bjorn, Lyngby (DK); Lars Christian Petersen, Hoersholm (DK)

Correspondence Address: Reza Green, Esq., Novo Nordisk Pharmaceuticals, Inc., 100 College Road West, Princeton, NJ, 08540, US

(21) Appl. No.: 10/617,500

(22) Filed: Jul. 11, 2003

Related U.S. Application Data
(60) Provisional application No. 60/404,567, filed on Aug. 19, 2002.

(30) Foreign Application Priority Data
Jul. 12, 2002 (DK) PA 2002 01100

(51) Int. Cl. A61K 38/37; C07K 14/755

(52) U.S. Cl. 514/12; 530/382

(57) ABSTRACT

This invention relates to novel compounds which bind to tissue factor and mediate a cytotoxic response.

### FIELD OF THE INVENTION

This invention relates to novel compounds that bind to and inhibit the activity of tissue factor and induce a cytotoxic response. The invention also relates to pharmaceutical compositions comprising the novel compounds as well as their use in the prophylaxis or treatment of diseases or disorders related to pathophysiological tissue factor (TF) functions including cancer, inflammation, atherosclerosis and ischemia/reperfusion.

### BACKGROUND OF THE INVENTION

Tissue Factor (TF) is a cellular transmembrane receptor for plasma coagulation factor VIIa (FVIIa) and formation of TF/FVIIa complexes on the cell surface triggers the coagulation cascade in vivo. The TF/FVIIa complex efficiently activates coagulation factors IX and X. The resultant protease factor Xa (FXa), activates prothrombin to thrombin, which in turn converts fibrinogen into a fibrin matrix.

Normally, TF is constitutively expressed on the surface of many extravascular cell types that are not in contact with the blood, such as fibroblasts, pericytes, smooth muscle cells and epithelial cells, but not on the surface of cells that come in contact with blood, such as endothelial cells and monocytes. However, TF is also expressed in various pathophysiological conditions where it is believed to be involved in progression of disease states within cancer, inflammation, atherosclerosis and ischemia/reperfusion. Thus, TF is now recognised as a target for therapeutic intervention in conditions associated with increased expression.

FVIIa is a two-chain, 50 kilodalton (kDa) vitamin-K dependent, plasma serine protease which participates in the complex regulation of in vivo haemostasis. FVIIa is generated from proteolysis of a single peptide bond from its single chain zymogen, Factor VII (FVII), which is present at approximately 0.5 μg/ml in plasma. The zymogen is catalytically inactive. The conversion of zymogen FVII into the activated two-chain molecule occurs by cleavage of an internal peptide bond. In the presence of calcium ions, FVIIa binds with high affinity to exposed TF, which acts as a cofactor for FVIIa, enhancing the proteolytic activation of its substrates FVII, Factor IX and FX.

In addition to its established role as an initiator of the coagulation process, TF was recently shown to function as a mediator of intracellular activities either by interactions of the cytoplasmic domain of TF with the cytoskeleton or by supporting the FVIIa-protease dependent signaling. Such activities may be responsible, at least partly, for the implicated role of TF in tumor development, metastasis and angiogenesis. Cellular exposure of TF activity is advantageous in a crisis of vascular damage but may be fatal when exposure is sustained as it is in these various diseased states. Thus, it is critical to regulate the expression of TF function in maintaining the health.

Inactivated FVII (FVIIai) is FVIIa modified in such a way that it is catalytically inactive. Thus, FVIIai is not able to catalyze the conversion of FX to FXa, but still able to bind tightly to TF in competition with active endogenous FVIIa and thereby inhibit the TF function.

International patent applications WO 92/15686, WO 94/27631, WO 96/12800, WO 97/47651 relates to FVIIai and the uses thereof. International patent applications WO 90/03390, WO 95/00541, WO 96/18653, and European Patent EP 500800 describes peptides derived from FVIIa having TF/FVIIa antagonist activity. International patent application WO 01/21661 relates to bivalent inhibitor of FVII and FXa.

Hu Z and Garen A (2001) Proc. Natl. Acad. Sci. USA 98; 12180-12185, Hu Z and Garen A (2000) Proc. Natl. Acad. Sci. USA 97; 9221-9225, Hu Z and Garen A (1999) Proc. Natl. Acad. Sci. USA 96; 8161-8166, and International patent application WO 0102439 relates to immunoconjugates which comprises the Fc region of a human IgG1 immunoglobulin and a mutant FVII polypeptide, that binds to TF but do not initiate blood clotting.

Furthermore, International patent application WO 98/03632 describes bivalent agonists having affinity for one or more G-coupled receptors, and Burgess, L. E. et al., Proc. Natl. Acad. Sci. USA 96, 8348-8352 (July 1999) describes “Potent selective non-peptidic inhibitors of human lung tryptase”.

There is still a need in the art for improved compounds that efficiently inhibit pathophysiological TF function at relatively low doses and which do not produce undesirable side effects. The present invention provides compounds that act specifically on pathophysiological TF function and at the same time elicit a cytotoxic response in a patient.

### SUMMARY OF THE INVENTION

The present invention relates in a broad aspect to TF antagonists.

In a first aspect, the present invention relates to a compound having the formula A—(LM)—C, wherein A comprises a TF antagonist; LM comprises an optional linker moiety; and C comprises a cytotoxic domain. In one embodiment, LM is present. In one embodiment, LM is absent.

In a second aspect, the present invention relates to a pharmaceutical composition comprising (i) an amount of the compound having the formula A—(LM)—C, wherein A comprises a TF antagonist; LM comprises an optional linker moiety; and C comprises a cytotoxic domain; and (ii) a pharmaceutically acceptable carrier or excipient.

In a third aspect, the present invention relates to a compound for use as a medicament having the formula A—(LM)—C, wherein A comprises a TF antagonist; LM comprises an optional linker moiety; and C comprises a cytotoxic domain; and wherein the compound binds to TF and inhibits TF function.

In a further aspect, the present invention relates to the use of a compound having the formula A—(LM)—C, wherein A comprises a TF antagonist; LM comprises an optional linker moiety; and C comprises a cytotoxic domain; for the manufacture of a medicament for preventing or treating disease or disorder associated with pathophysiological TF function.

In a further aspect, the present invention relates to a method for preventing or treating disease or disorder associated with pathophysiological TF function, said method comprising contacting a TF presenting cell with a compound having the formula A—(LM)—C, wherein A comprises a TF antagonist; LM comprises an optional linker moiety; and C comprises a cytotoxic domain.

### BRIEF DESCRIPTION OF THE DRAWINGS

FIG. 1 illustrates the amino acid sequence of human Factor VIIa (SEQ ID NO:I) and the amino acid sequences of two pro-apoptotic peptides, SEQ ID NO:2 and SEQ ID NO:3.

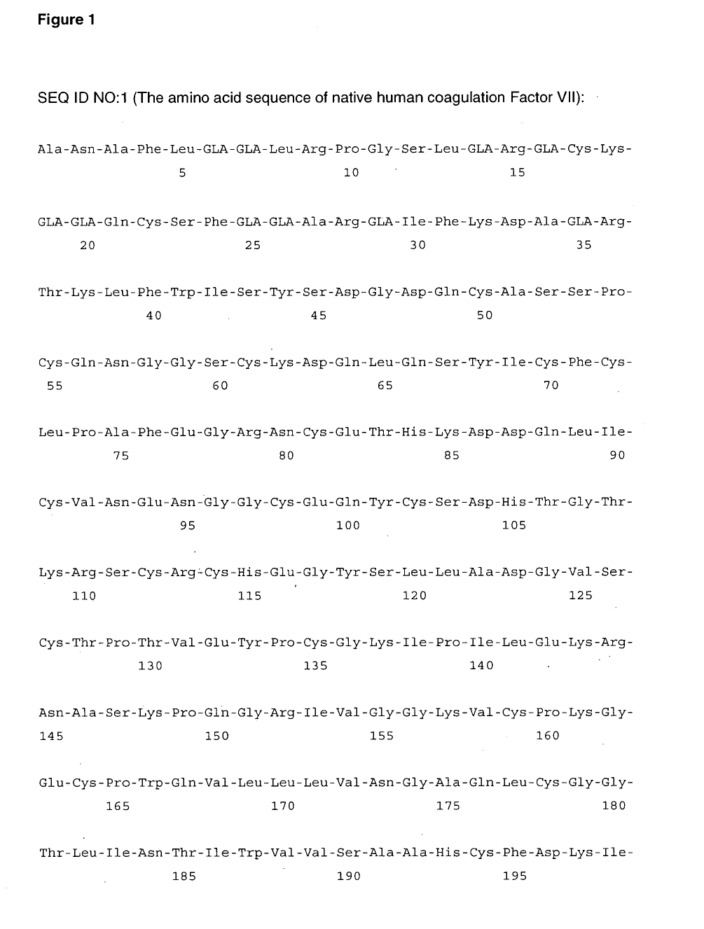

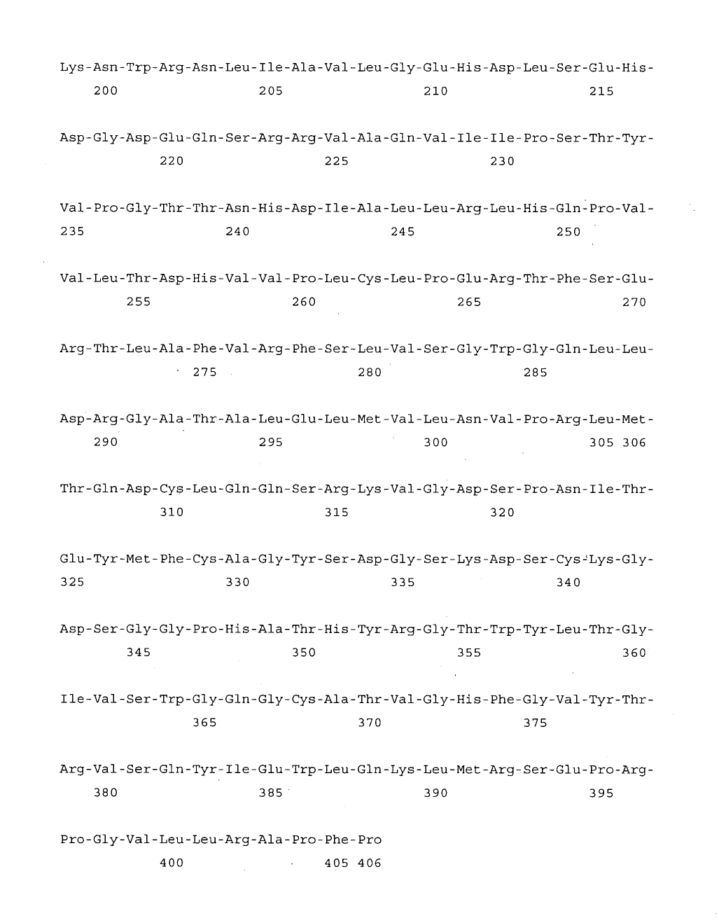

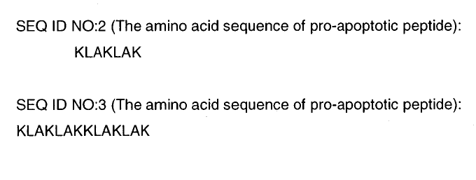

### DETAILED DESCRIPTION OF THE INVENTION

The present invention relates to conjugates of (i) a TF antagonist and (ii) a domain that elicits a cytotoxic response. The conjugate binds TF with high affinity and specificity but does not initiate blood coagulation. In one embodiment of the present invention, the TF antagonist comprises a factor FVIIa polypeptide that has been chemically inactivated in the active site. In another embodiment of the present invention, the TF antagonist comprises an antibody against TF. In one embodiment, the antibody is a monoclonal antibody. In one embodiment, the antibody is a human monoclonal antibody. In one embodiment, the antibody is an antibody against human TF.

Methods of preparing human antibodies against human TF are described in Danish patent application number PA 2001 01437.

The conjugates contain a functional domain that elicits a cytotoxic response, which destroys or kills the target cells in the patient.

The terms “TF antagonist” or “TF antagonists”, as used herein is intended to mean any compound that binds directly to TF and inhibits TF-mediated FVIIa activity.

The term “TF-mediated FVIIa activity”, as used herein means any TF-dependent activity. The term is intended to include both a TF-mediated coagulation activity and a signaling activity mediated by TF, e.g. TF-mediated MAPK signaling. In one embodiment, the TF-mediated FVIIa activity is MAPK signaling.

The term “TF-mediated MAPK signaling” is intended to mean a cascade of intracellular events that mediate activation of Mitogen-Activated-Protein-Kinase (MAPK) or homologues thereof in response to the binding of a FVII polypeptide to TF. Three distinct groups of MAP kinases have been identified in mammalian cells: 1) extracellular-regulated kinase (Erk1/2 or p44/42), 2) c-Jun N-terminal kinase (JNK) and 3) p38 kinase. The Erk1/2 pathway involves phosphorylation of Erk 1 (p 44) and/or Erk 2 (p 42). Activated MAP kinases e.g. p44/42 MAPK can translocate to the nucleus where they can phosphorylate and activate transcription factors including (Elk 1) and signal transducers and activators of transcription (Stat). Erk1/2 can also phosphorylate the kinase p90RSK in the cytoplasm or in the nucleus, and p90RSK then can activate several transcription factors. MAPK signaling may be measured as described in assay 5.

The term “protein kinase” is intended to indicate an enzyme that is capable of phosphorylating serine and/or threonine and/or tyrosine in peptides and/or proteins.

The term “FVIIa-induced activation of the MAPK signaling” is intended-to indicate that FVIIa binds to TF in a mammalian cell and thereby induce MAPK signaling.

The term “TF-mediated coagulation activity” means coagulation initiated by TF through the formation of the TF/FVIIa complex and its activation of FIX and Factor X to FIXa and FXa, respectively. TF-mediated coagulation activity is measured in a FXa generation assay. The term “FXa generation assay” as used herein is intended to mean any assay where activation of FX is measured in a sample comprising TF, FVIIa, FX, calcium and phospholipids. An example of a FXa generation assay is described in assay 1.

Examples of TF antagonist include, but are not limited to, FVIIai and inhibitory antibodies against TF.

Currently no TF antagonists have been developed and marketed for therapeutic use in humans. Known therapeutic strategies include monoclonal antibodies, catalytically impaired FVIIa mutants and chemical inactivated FVIIa. Native FVIIa binds TF with high affinity and most mutants with amino acid substitutions and monoclonal antibodies is expected to bind with equal or less affinity. The low binding affinity for TF may limit their effective use in the clinic. Chemically inactivated FVIIa has been reported to have a modest increase in the affinity for TF as compared to native FVIIa.

The reported inactive mutants of FVIIa as well as the chemically inactivated FVIIa are expected to have short half lives compared to that of circulating native FVII, i.e. 2-3 hours, which may limit their effective use in the clinic.

The present invention relates to TF antagonists conjugated to a cytotoxic domain. It is to be understood, that the conjugate binds to and kills or arrest the growth of the TF presenting cells. The terms “TF presenting cell” or “TF presenting cells” as used herein refers to the presence of TF protein on a cell surface plasma membrane. The cell membrane, where TF is located may be the cell, where TF was synthezised by protein synthesis or it may be a cell, that contain shedded TF protein synthezised by another cell.

The inactivation of the FVIIa proteolytic activity is obtained in vitro by covalent active site inhibitors e.g. chloromethyl ketones. The conjugate has very high affinity for TF due to the increased affinity of the chemically inactivated binding domain as compared to the binding of native FVII. The high affinity provides a more efficacious and safe treatment of a patient in need thereof. The conjugate may also have a higher affinity for TF due an avidity effect in dimers, trimers or other multimers with multible TF binding sites.

Cytotoxic domains include, but are not limited to, peptides that directly mediate mitochondrial cytochrome C release and apoptosis, such as, e.g., (KLAKLAK)₂ including both enantiomers (Ellerby et al. Nature medicine 5, (9) 1032-1038, 1999), any cytostatic agent that is capable of inducing cell ablation, and any compound that can alleviate resistance to cell death signals initiated by exogenous or endogenous compounds.

In a preferred embodiment, the present invention relates to chemically inactivated FVII molecules in which the active site is covalently modified by any of the following:

1) A covalent active site inhibitor for delivery of cytotoxic compounds, which may be released by reduction. Cytotoxic compounds include any cytostatic agent that may be coupled to an active site inhibitor and be released in a functional form following transfer to a reducing environment, e.g., cytoplasm or lysosomes.

2) A covalent active site inhibitor for delivery of cytotoxic compounds released by a specific hydrolase either inside the cell or on the cell surface, principally (but not limited to) lysosomal proteases, e.g., Cathepsin B, lipases and the like. Here the inhibitor may be coupled to any substrate from which a cytotoxic compound may be released in a functional form by hydrolysis.

3) A covalent active site inhibitor for delivery of a compound, which may be activated at the desired site of action by an exogenous stimuli, e.g. photodynamic compounds activated by an electromagnetic field, e.g. light.

4) A covalent active site inhibitor for delivery of radionuclides. In this scenario, radionuclides selectively accumulate inside the targeted cell.

While full length FVII does represent the preferred embodiment for drug delivery in 1, 2 and 3, this does not exclude the use of FVII (des-Gla) or any other TF-binding FVII derived protein including truncated forms, analogs, derivatives and fusion proteins (monomers, homo- or heterodimers or multimers). The different affinity of such molecules for TF can provide a method for reducing the potentially undesirable effect of a cytotoxic compound on general haemostasis.

The term “disease or disorder associated with pathophysiological TF function” as used herein means any disease or disorder, where TF is involved. This includes, but are not limited to, diseases or disorders related to TF-mediated coagulation activity, thrombotic or coagulopathic related diseases or disorders, or diseases or disorders such as inflammatory responses and chronic thromboembolic diseases or disorders associated with fibrin formation, including vascular disorders such as deep venous thrombosis, arterial thrombosis, post surgical thrombosis, coronary artery bypass graft (CABG), percutaneous transdermal coronary angioplastry (PTCA), stroke, cancer, tumor metastasis, angiogenesis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases. The diseases or disorders associated with pathophysiological TF function are not limited to in vivo coagulopatic disorders such as those named above, but also include ex vivo TF/FVIIa related processes such as coagulation that may result from the extracorporeal circulation of blood, including blood removed in-line from a patient in such processes as dialysis procedures, blood filtration, or blood bypass during surgery.

“Treatment” means the administration of an effective amount of a therapeutically active compound of the invention with the purpose of preventing any symptoms or disease state to develop or with the purpose of curing or easing such symptoms or disease states already developed. The term “treatment” is thus meant to include prophylactic treatment.

The terms “cancer or “tumor” are to be understood as referring to all forms of neo-plastic cell growth, including tumors of the lung, liver, blood cells (leukaemias), skin, pancreas, colon, prostate, uterus or breast.

In one embodiment of the invention, the disease or disorder associated with pathophysiological TF function is deep venous thrombosis, arterial thrombosis, post surgical thrombosis, coronary artery bypass graft (CABG), percutaneous transdermal coronary angioplastry (PTCA), stroke, cancer, tumour metastasis, angiogenesis, ischemia/reperfusion, rheumatoid arthritis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis.

In one embodiment of the invention, A in the compound having the formula A—(LM)—C is an inactive FVIIa polypeptide.

In a further embodiment of the invention, A in the compound having the formula A—(LM)—C is native human FVIIa or a fragment thereof catalytically inactivated in the active site.

In a further embodiment of the invention, A in the compound having the formula A—(LM)—C is native human FVIIa catalytically inactivated in the active site.

In a further embodiment of the invention, C or (LM)—C in the compound having the formula A—(LM)—C is conjugated to the active site of the FVIIa polypeptide.

In a further embodiment of the invention, A in the compound having the formula A—(LM)—C is an inactive FVIIa polypeptide catalytically inactivated in the active site with a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethylketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone, and Dansyl-D-Glu-Gly-Arg chloromethylketone.

In a further embodiment of the invention, LM in the compound having the formula A—(LM)—C comprises a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone, and Dansyl-D-Glu-Gly-Arg chloromethylketone, wherein the inactive FVIIa polypeptide is catalytically inactivated in the active site with said chloromethyl ketone inhibitor.

In a further embodiment of the invention, A in the compound having the formula A—(LM)—C is an antibody against TF.

In a further embodiment of the invention, A in the compound having the formula A—(LM)—C is a human monoclonal antibody against human TF.

In a further embodiment of the invention, C in the compound having the formula A—(LM)—C is selected from the group consisting of protein ionophores, cytostatica, chemotherapeutic compound, compounds which induce apoptosis, compound containing radionuclides, antisense nucleotide molecules independent selected from the group consisting of PNAs, DNAs, RNAs and LNAs. In one embodiment, C in the compound having the formula A—(LM)—C is a cytostatica. In one embodiment, C in the compound having the formula A—(LM)—C is a chemotherapeutic compound. In one embodiment, C in the compound having the formula A—(LM)—C is a compound containing radionuclides. In one embodiment, C in the compound having the formula A—(LM)—C is an antisense nucleotide molecule. In a specific embodiment, C in the compound having the formula A—(LM)—C is melphalan. In another specific embodiment, C in the compound having the formula A—(LM)—C comprises I¹²⁵.

In a further embodiment of the invention, C in the compound having the formula A—(LM)—C comprises a cytotoxic protein or peptide.

In a further embodiment of the invention, C in the compound having the formula A—(LM)—C is comprises the amino acid sequence (KLAKLAK)ₙ, wherein n is selected from the group consisting of 1, 2, 3, 4, 5, 6, 7, and 8. In one embodiment n is 2.

In a further embodiment of the invention, C in the compound having the formula A—(LM)—C has the amino acid sequence (KLAKLAK)₂

In a further embodiment of the invention, C or (LM)—C in the compound having the formula A—(LM)—C is conjugated to an oligosaccharide side chain present on A.

In a further embodiment of the invention, C or (LM)—C in the compound having the formula A—(LM)—C is conjugated to a free sulfhydryl group present on A.

In a further embodiment, the compound having the formula A—(LM)—C comprises more than one binding site for TF. In one embodiment, the compound is a dimer. In one embodiment, the compound is a trimer. In one embodiment, the compound is a tetramer. In one embodiment, the compound is a pentamer. In one embodiment, the compound is a hexamer.

In a further embodiment of the invention, LM in the compound having the formula A—(LM)—C comprises an amino acid sequence.

In a further embodiment of the invention, LM in the compound having the formula A—(LM)—C comprises the amino acid sequence Gly-Gly.

In a further embodiment of the invention, LM in the compound having the formula A—(LM)—C comprises a molecule selected from the group consisting of straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl.

In a further embodiment of the invention, LM in the compound having the formula A—(LM)—C comprises a chemical bond, which can broken by chemical reduction.

In a further embodiment of the invention, LM in the compound having the formula A—(LM)—C comprises a disulfide bond. In one embodiment, the disulfide bond is between two cysteines.

In a further embodiment of the invention, LM in the compound having the formula A—(LM)—C comprises a cleavage site for enzyme hydrolysis. In one embodiment, the enzyme is a lipase. In another embodiment, the enzyme is a protease.

In a further embodiment of the invention, LM in the compound having the formula A—(LM)—C comprises a cleavage site for protease hydrolysis, wherein the protease is selected from the group consisting of cathepsin B, cathepsin D, cathepsin E, cathepsin G, cathepsin H, cathepsin L, cathepsin N, cathepsin S, cathepsin T, cathepsin K, and legumain. In a specific embodiment, the protease is cathepsin B.

In a further embodiment of the invention, LM in the compound having the formula A—(LM)—C comprises the amino acid sequence Phe-Arg.

The terms “cytotoxic domain” or “cytotoxic compound” as used herein refers to a substance that inhibits or prevents the function of cells and/or causes destruction of cells. The terms “cytotoxic domain” and “cytotoxic compound” may be used interchangeably. The term is intended to include radioactive isotopes or radionuclides (e.g. I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic compound” is a chemical compound useful in the treatment of cancer. Examples of chemotherapeutic compounds include adriamycin, doxorubicin, epirubicin, 5-fluorouracil, cytosine arabinoside (“Ara-C”), cyclophosphamide, thiotepa, busulfan, cytoxin, taxoids, e.g. paclitaxel (TAXOL™, Bristol-Myers Squibb Oncology, Princeton, N.J.), and doxetaxel, toxotere, methotrexate, cisplatin, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), melphalan and other related nitrogen mustards. Also included in this definition are hormonal compounds that act to regulate or inhibit hormone action on tumors, such as tamoxifen and onapristone.

Cytotoxic compounds include, but are not limited to: toxins; drugs; enzymes; cytokines; radionuclides; photodynamic compounds; and molecules which induce apoptosis (e.g., Fas ligand or 2-methoxyestradiol). Toxins may include, without limitation, ricin A chain, mutant Pseudomonas exotoxins, diphtheria toxoid, streptonigrin, boamycin, saporin, gelonin, and pokeweed antiviral protein. Drugs may include of cytotoxic drugs including, but not limited to, fludarabine, chlorambucil, daunorubicin, doxorubicin (e.g., in liposomes), cisplatin, bleomycin, melphalan, mitomycin-C, and methotrexate. Due to the sensitivity of B cells to radiation, radionuclides may include, but are not limited to, proteins labeled with radiometals such as yttrium which emits a high energy beta particle, and I¹²⁵ that emits Auger electrons, that may be absorbed by adjacent TF presenting cells. Photodynamic compounds may include therapeutically effective amounts of porphyrins and their derivatives. The methods for coupling ligands or targeting molecules with therapeutic compounds are well known to those skilled in the art (See, for example, conjugates as reviewed by Ghetie et al., 1994, Pharmacol. Ther. 63:209-34; U.S. Pat. No. 5,789,554, the disclosure of which is herein incorporated by reference). Often such methods utilize one of several available hetero-bifunctional reagents used for coupling or linking molecules.

Cytotoxic compounds suitable for use herein include, without limitation, conventional chemotherapeutics, such as vinblastine, anthracycline antitumor antibiotics including doxorubicin, 2-pyrrolino-doxorubicin, Doxorubicin hydrochloride (Adriamycin) (Schally V A and Nagy A (1999) Eur J Endocrinol 141, 1-14, Vasey P A et al (1999) Clin Cancer Res 5, 83-94), bleomycin, methotrexate, 5-fluorouracil, 6-thioguanine, cytarabine, cyclophosphamide (N,N-bis-(beta-Chlorethyl)-amino-1-oxa-3-aza-2-phosphocyclohexane-2-oxide) and cisplatinum, as well as other conventional chemotherapeutics as described in Cancer: Principles and Practice of Oncology, 2d ed., V. T. DeVita, Jr., S. Hellman, S. A. Rosenberg, J.B. Lippincott Co., Philadelphia, Pa., 1985, Chapter 14. Another suitable cytotoxic compound within the present invention is a trichothecene. Trichothecenes are drugs produced by soil fungi of the class Fungi imperfecti or isolated from Baccharus megapotamica (Bamburg, J. R. Proc. Molec. Subcell. Biol. 8:41-110, 1983; Jarvis & Mazzola, Acc. Chem. Res. 15:338-395, 1982). They appear to be the most toxic molecules that contain only carbon, hydrogen and oxygen (Tamm, C. Fortschr. Chem. Org. Naturst. 31:61-117, 1974). They are all reported to act at the level of the ribosome as inhibitors of protein synthesis at the initiation, elongation, or termination phases.

There are two broad classes of trichothecenes: those that have only a central sesquiterpenoid structure and those that have an additional macrocyclic ring (simple and macrocyclic trichothecenes, respectively). The simple trichothecenes may be subdivided into three groups (i.e., Group A, B, and C) as described in U.S. Pat. Nos. 4,744,981 and 4,906,452 (incorporated herein by reference). Representative non-limiting examples of Group A simple trichothecenes include: Scirpene, Roridin C, dihydrotrichothecene, Scirpen-4, 8-diol, Verrucarol, Scirpentriol, T-2 tetraol, pentahydroxyscirpene, 4-deacetylneosolaniol, trichodermin, deacetylcalonectrin, calonectrin, diacetylverrucarol, 4-monoacetoxyscirpenol, 4,15-diacetoxyscirpenol, 7-hydroxydiacetoxyscirpenol, 8-hydroxydiacetoxyscirpenol (Neosolaniol), 7,8-dihydroxydiacetoxyscirpenol, 7-hydroxy-8-acetyldiacetoxyscirpenol, 8-acetylneosolaniol, NT-1, NT-2, HT-2, T-2, and acetyl T-2 toxin. Representative non-limiting examples of Group B simple trichothecenes include: Trichothecolone, Trichothecin, deoxynivalenol, 3-acetyldeoxynivalenol, 5-acetyldeoxynivalenol, 3,15-diacetyldeoxynivalenol, Nivalenol, 4-acetylnivalenol (Fusarenon-X) 4,15-idacetylnivalenol, 4,7,15-triacetylnivalenol, and tetra-acetylnivalenol. Representative non-limiting examples of Group C simple trichothecenes include: Crotocol and Crotocin. Representative non-limiting macrocyclic trichothecenes include Verrucarin A, Verrucarin B, Verrucarin J (Satratoxin C), Roridin A, Roridin D, Roridin E (Satratoxin D), Roridin H, Satratoxin F, Satratoxin G, Satratoxin H; Vertisporin, Mytoxin A, Mytoxin C, Mytoxin B, Myrotoxin A, Myrotoxin B, Myrotoxin C, Myrotoxin D, Roritoxin A, Roritoxin B, and Roritoxin D. In addition, the general “trichothecene” sesquiterpenoid ring structure is also present in compounds termed “baccharins” isolated from the higher plant Baccharis megapotamica, and these are described in the literature, for instance as disclosed by Jarvis et al. (Chemistry of Alleopathy, ACS Symposium Series No. 268: ed. A. C. Thompson, 1984, pp. 149-159).

Other suitable cytotoxic compound within the present invention include, without limitation, N,N-cis(2-chloroethyl)N-nitroso-urea (BCNU), D-myo-inositol-1,2,6-trisphosphate, Melphalan (p-Di-(2-chloroethyl)-amino-L-phenylalanine), Procarbazine (p-(N′-Methyl-hydrazinomethyl)-N-isopropyl-benzamide), Dactinomycin (Actinomycin D), Polyestradiolphosphate, thalidomid, temozolomide, mitozolomide, mercaptopurine, N-methylformamide, 2-amino-1,3,4-thiadiazole, hexamethylmelamine, gallium nitrate, 3% thymidine, dichloromethotrexate, mitoguazone, suramin, bromodeoxyuridine, iododeoxyuridine, semustine, 1-(2-chloroethyl)-3-(2,6-dioxo-3-piperidyl)-1-nitrosourea, N,N′-hexamethylene-bis-acetamide, azacitidine, dibromodulcitol, Erwinia asparaginase, ifosfamide, 2-mercaptoethane sulfonate, teniposide, taxol, 3-deazauridine, soluble Baker's antifol, homoharringtonine, cyclocytidine, acivicin, ICRF-187, spiromustine, levamisole, chlorozotocin, aziridinyl benzoquinone, spirogermanium, aclarubicin, pentostatin, PALA, carboplatin, amsacrine, caracemide, iproplatin, misonidazole, dihydro-5-azacytidine, 4′-deoxy-doxorubicin, menogaril, triciribine phosphate, fazarabine, tiazofurin, teroxirone, ethiofos, N-(2-hydroxyethyl)-2-nitro-1H-imidazole-1-acetamide, mitoxantrone, acodazole, amonafide, fludarabine phosphate, pibenzimol, didemnin B, merbarone, dihydrolenperone, flavone-8-acetic acid, oxantrazole, ipomeanol, trimetrexate, deoxyspergualin, echinomycin, and dideoxycytidine (see NCI Investigational Drugs, Pharmaceutical Data 1987, NIH Publication No. 88-2141, Revised November 1987) are also preferred.

In one embodiment of the invention, the cytotoxic domain stimulates the production of free radicals NO*, O2*.

In one embodiment of the invention, the cytotoxic domain stimulates apoptosis by regulation of p53, superoxiddismutase, phospholipase C, cyclooxygenase 2, caspase-associated recruitment domains (CARD).

In one embodiment of the invention, the cytotoxic domain is selected from the group consisting of an cyclooxygenase 2 inhibitor, apoptin, chicken Anemia Virus (CAV), (e.g. CAV protein VP1, VP2 and VP3 (apoptin) U.S. Pat. No. 5,981,502), Sulforaphane (SUL).

Radionuclides useful within the present invention include, without limitation, gamma-emitters, positron-emitters, Auger electron-emitters, X-ray emitters and fluorescence-emitters, with beta- or alpha-emitters preferred for therapeutic use. Radionuclides are well-known in the art and include 123-I, 125-I, 130-I, 131-I, 133-I, 135-I 47-Sc, 72-As, 72-Se, 90-Y, 88-Y, 97-Ru, 100-Pd, 101m-Rh, 119-Sb, 128-Ba, 197-Hg, 211-At, 212-Bi, 153-Sm, 169-Eu, 212-Pb, 109-Pd, 111-In, 67-Ga, 68-Ga, 64-Cu, 67-Cu, 75-Br, 76-Br, 77-Br, 99m-Tc. 11-C, 13-N, 15-O, 166-Ho and 18-F. Preferred therapeutic radionuclides include 188-Re, 186-Re, 203-Pb, 212-Pb, 212-Bi, 109-Pd, 64-Cu, 67-Cu, 90-Y, 125-I, 131-I, 77-Br, 211-At, 97-Ru, 105-Rh, 198-Au and 199-Ag, 166-Ho or 177-Lu.

In one embodiment of the invention, the TF antagonist comprises gadophrin e.g. gadophrin-2 (previously referred as bis-gadolinium-mesoporhyrin) (Pislaru S V. et al., Circulation, 99 (5) pp. 690-696, 1999).

The terms “Nucleic acid sequence” or “nucleotide sequence” as used herein refer to an oligonucleotide, nucleotide, or polynucleotide, and fragments or portions thereof, and to DNA or RNA of genomic or synthetic origin which may be single- or double-stranded, and represent the sense or antisense strand. Similarly, “amino acid sequence” as used herein refers to an oligopeptide, peptide, polypeptide, or protein sequence, and fragments or portions thereof, and to naturally occurring or synthetic molecules.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

The terms “PNA” or “Peptide nucleic acid”, as used herein, refer to a molecule which comprises an oligomer to which an amino acid residue, such as lysine, and an amino group have been added. These small molecules, also designated anti-gene agents, stop transcript elongation by binding to their complementary strand of nucleic acid (Nielsen, P. E. et al. (1993) Anticancer Drug Des. 8:53-63).

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated. The designation “negative” is sometimes used in reference to the antisense strand, and “positive” is sometimes used in reference to the sense strand.

A further preferred antisense molecule includes Locked Nucleic Acids (LNAS) in which the 2′-hydroxyl group is linked to the 3′ or 4′ carbon atom of the sugar ring thereby forming a bicyclic sugar moiety. The linkage is preferably a methylene (—CH₂—).sub.n group bridging the 2′ oxygen atom and the 4′ carbon atom wherein n is 1 or 2. LNAs and preparation thereof are described in WO 98/39352 and WO 99/14226.

The terms “FVIIa polypeptide” or “FVIIa polypeptides” as used herein means native Factor VIIa, as well as equivalents of Factor VIIa that contain one or more amino acid sequence alterations relative to native Factor VIIa (i.e., Factor VII variants), and/or contain truncated amino acid sequences relative to native Factor VIIa (i.e., Factor VIIa fragments). Such equivalents may exhibit different properties relative to native Factor VIIa, including stability, phospholipid binding, altered specific proteolytic activity, and the like.

As used herein, “Factor VII equivalent” encompasses, without limitation, equivalents of Factor VIIa exhibiting TF binding activity. The term “TF binding activity” as used herein means the ability of a FVIIa polypeptide or TF antagonist to inhibit the binding of recombinant human ¹²⁵I-FVIIa to cell surface human TF. The TF binding activity may be measured as described in Assay 3.

Factor VII equivalents also include proteolytically inactive variants of FVIIa. In one embodiment of the invention, the FVIIa polypeptide is human FVIIa, which has an amino acid substitution of the lysine corresponding to position 341 of SEQ ID NO: 1.

In one embodiment of the invention, the FVIIa polypeptide is human FVIIa that has an amino acid substitution of the serine corresponding to position 344 of SEQ ID NO: 1.

In one embodiment of the invention, the FVIIa polypeptide is human FVIIa that has an amino acid substitution of the aspartic acid corresponding to position 242 of SEQ ID NO: 1.

In one embodiment of the invention the FVIIa polypeptide is human FVIIa that has an amino acid substitution of the histidine corresponding to position 193 of SEQ ID NO: 1.

In one embodiment, the FVIIa polypeptide is FVII-(K341A)

In one embodiment, the FVIIa polypeptide is FVII-(S344A)

In one embodiment, the FVIIa polypeptide is FVII-(D242A)

In one embodiment, the FVIIa polypeptide is FVII-(H193A)

The terminology for specific amino acid substitutions used herein is as follows. The first letter represent the amino acid naturally present at a position of SEQ ID NO: 1. The following number represents the position in SEQ ID NO: 1. The second letter represents the different amino acid substituting for the natural amino acid. An example is FVII-(K341A), where a lysine at position 341 of SEQ ID NO: 1 is replaced by an alanine. In another example, FVII-(K341A/S344A), the lysine at position 341 of SEQ ID NO: 1 is replaced by an alanine and the serine in position 344 of SEQ ID NO: 1 is replaced by an alanine in the same Factor VII polypeptide.

The terms “Factor VII” or “FVII” are intended to mean Factor VII polypeptides in their uncleaved (zymogen) form.

The terms “Factor VIIa” or “FVIIa” are intended to mean native bioactive forms of FVII. Typically, FVII is cleaved between residues 152 and 153 to yield FVIIa. The term “Factor VIIa” is also intended to encompass, without limitation, polypeptides having the amino acid sequence 1-406 of wild-type human Factor VIIa (as disclosed in U.S. Pat. No. 4,784,950), as well as wild-type Factor VIIa derived from other species, such as, e.g., bovine, porcine, canine, murine, and salmon Factor VIIa. It further encompasses natural allelic variations of Factor VIIa that may exist and occur from one individual to another. Also, degree and location of glycosylation or other post-translation modifications may vary depending on the chosen host cells and the nature of the host cellular environment.

The terms “variant” or “variants”, as used herein, is intended to designate human Factor VII having the sequence of SEQ ID NO: 1, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. In one embodiment of the invention the variant has a total amont of amino acid substitutions and/or additions and/or deletions independently selected from the group consisting of 1, 2, 3, 4, 5, 6, 7, 8, 9, and 10.

The ability of any particular conjugate of a TF antagonist and a cytotoxic compound to mediate lysis of the tumor cell target can be assayed. The tumor cells of interest are grown and labeled in vivo or in vitro; the cytotoxic TF antagonist conjugate is added to the tumor cell culture. Cytolysis of the target tumor cells is detected by the release of label from the lysed cells. The cytotoxic TF antagonist conjugate that is capable of mediating cell ablation, lysis or apoptosis in the in vitro test can then be used therapeutically in that particular patient.

The term “active site” and the like when used herein with reference to FVIIa refer to the catalytic and zymogen substrate binding site, including the “S₁” site of FVIIa as that term is defined by Schecter, I. and Berger, A., (1967) Biochem. Biophys. Res. Commun. 7:157-162.

The term “TF-mediated coagulation activity” means coagulation initiated by TF through the formation of the TF/FVIIa complex and its activation of FIX and Factor X to FIXa and FXa, respectively. TF-mediated coagulation activity is measured in a FXa generation assay. The term “FXa generation assay” as used herein is intended to mean any assay where activation of FX is measured in a sample comprising TF, FVIIa, FX, calcium and phospholipids. An example of a FXa generation assay is described in assay 1.

A TF/FVIIa mediated or associated process or event, or a process or event associated with TF-mediated coagulation activity, is any event, which requires the presence of TF/FVIIa.

Such processes or events include, but are not limited to, formation of fibrin which leads to thrombus formation; platelet deposition; proliferation of smooth muscle cells (SMCs) in the vessel wall, such as, for example, in intimal hyperplasia or restenosis, which is thought to result from a complex interaction of biological processes including platelet deposition and thrombus formation, release of chemotactic and mitogenic factors, and the migration and proliferation of vascular smooth muscle cells into the intima of an arterial segment; and deleterious events associated with post-ischemic reperfusion, such as, for example, in patients with acute myocardial infarction undergoing coronary thrombolysis.

The no-reflow phenomenon, that is, lack of uniform perfusion to the microvasculature of a previously ischemic tissue has been described for the first time by Krug et al., (Circ. Res. 1966; 19:57-62).

The general mechanism of blood clot formation is reviewed by Ganong, in Review of Medical Physiology, 13^{th} ed., Lange, Los Altos Calif., pp 411-414 (1987). Coagulation requires the confluence of two processes, the production of thrombin which induces platelet aggregation and the formation of fribrin which renders the platelet plug stable. The process comprises several stages each requiring the presence of discrete proenzymes and profactors. The process ends in fibrin crosslinking and thrombus formation. Fibrinogen is converted to fibrin by the action of thrombin. Thrombin, in turn, is formed by the proteolytic cleavage of prothrombin. This proteolysis is effected by FXa which binds to the surface of activated platelets and in the presence of FVa and calcium, cleaves prothrombin. TF/FVIIa is required for the proteolytic activation of FX by the extrinsic pathway of coagulation. Therefore, a process mediated by or associated with TF/FVIIa, or an TF-mediated coagulation activity includes any step in the coagulation cascade from the formation of the TF/FVIIa complex to the formation of a fibrin platelet clot and which initially requires the presence of TF/FVIIa. For example, the TF/FVIIa complex initiates the extrinsic pathway by activation of FX to FXa, FIX to FIXa, and additional FVII to FVIIa. TF/FVIIa mediated or associated process, or TF-mediated coagulation activity can be conveniently measured employing standard assays such as those described in Roy, S., (1991) J. Biol. Chem. 266:4665-4668, and O'Brien, D. et al., (1988) J. Clin. Invest. 82:206-212 for the conversion of FX to FXa in the presence of TF/FVIIa and other necessary reagents.

The term “disease or disorder associated with pathophysiological TF function” as used herein means any disease or disorder, where TF is involved. This includes, but is not limited to, diseases or disorders related to TF-mediated coagulation activity, thrombotic or coagulopathic related diseases or disorders or diseases or disorders such as inflammatory responses and chronic thromboembolic diseases or disorders associated with fibrin formation, including vascular disorders such as deep venous thrombosis, arterial thrombosis, post surgical thrombosis, coronary artery bypass graft (CABG), percutaneous transdermal coronary angioplastry (PTCA), stroke, tumour metastasis, angiogenesis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, or the prophylactic treatment of mammals with atherosclerotic vessels at risk for thrombosis, and other diseases. The disease or disorder associated with pathophysiological TF function is not limited to in vivo coagulopathic disorders such as those named above, but also includes ex vivo TF/FVIIa related processes such as coagulation that may result from the extracorporeal circulation of blood, including blood removed in-line from a patient in such processes as dialysis procedures, blood filtration, or blood bypass during surgery.

“Treatment” means the administration of an effective amount of a therapeutically active compound of the invention with the purpose of preventing any symptoms or disease state to develop or with the purpose of curing or easing such symptoms or disease states already developed. The term “treatment” is thus meant to include prophylactic treatment.

It should be noted that peptides, proteins and amino acids as used herein can comprise or refer to “natural”, i.e., naturally occurring amino acids as well as “non.classical” D-amino acids including, but not limited to, the D-isomers of the common amino acids, α-isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, Nα-methyl amino acids, and amino acid analogues in general. In addition, the amino acids can include Abu, 2-amino butyric acid; γ-Abu, 4-aminobutyric acid; ε-Ahx, 6-aminohexanoic acid; Aib, 2-amino-isobutyric acid; β-Ala, 3-aminopropionic acid; Orn, ornithine; Hyp, trans-hydroxyproline; Nle, norleucine; Nva, norvaline.

The three-letter indication “GLA” as used herein means 4-carboxyglutamic acid (γ-carboxyglutamate).

By “catalytically inactivated in the active site of the FVIIa polypeptide” is meant that a FVIIa inhibitor is bound to the FVIIa polypeptide and decreases or prevents the FVIIa-catalysed conversion of FX to FXa. A FVIIa inhibitor may be identified as a substance, which reduces the amidolytic activity by at least 50% at a concentration of the substance at 400 μM in the FVIIa amidolytic assay described by Persson et al. (Persson et al., J. Biol. Chem. 272: 19919-19924 (1997)). Preferred are substances reducing the amidolytic activity by at least 50% at a concentration of the substance at 300 μM; more preferred are substances reducing the amidolytic activity by at least 50% at a concentration of the substance at 200 μM.

The “FVIIa inhibitor” may be selected from any one of several groups of FVIIa directed inhibitors. Such inhibitors are broadly categorised for the purpose of the present invention into i) inhibitors which reversibly bind to FVIIa and are cleavable by FVIIa, ii) inhibitors which reversibly bind to FVIIa but cannot be cleaved, and iii) inhibitors which irreversibly bind to FVIIa. For a review of inhibitors of serine proteases see Proteinase Inhibitors (Research Monographs in cell and Tissue Physiology; v. 12) Elsevier Science Publishing Co., Inc., New York (1990).

The FVIIa inhibitor moiety may also be an irreversible FVIIa serine protease inhibitor. Such irreversible active site inhibitors generally form covalent bonds with the protease active site. Such irreversible inhibitors include, but are not limited to, general serine protease inhibitors such as peptide chloromethylketones (see, Williams et al., J. Biol. Chem. 264:7536-7540 (1989)) or peptidyl cloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosyllysylchloromethyl ketone (TLCK); nitrophenyl-sulphonates and related compounds; heterocyclic protease inhibitors such as isocoumarines, and coumarins.

Examples of peptidic irreversible FVIIa inhibitors include, but are not limited to, Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone.

Examples of FVIIa inhibitors also include benzoxazinones or heterocyclic analogues thereof such as described in PCT/DK99/00138.

Examples of other FVIIa inhibitors include, but are not limited to, small peptides such as for example Phe-Phe-Arg, D-Phe-Phe-Arg, Phe-Phe-Arg, D-Phe-Phe-Arg, Phe-Pro-Arg, D-Phe-Pro-Arg, Phe-Pro-Arg, D-Phe-Pro-Arg, L- and D-Glu-Gly-Arg; peptidomimetics; benzamidine systems; heterocyclic structures substituted with one or more amidino groups; aromatic or heteroaromatic systems substituted with one or more C(═NH)NHR groups in which R is H, C₁₋₃alkyl, OH or a group which is easily split of in vivo.

By “linker moiety” or “LM” is meant any biocompatible molecule functioning as a means to link the cytotoxic domain to the TF antagonist. The terms “linker”, linker part”, “linker part B”, “spacer” as used herein all refers to parts of the LM. The TF antagonist and the cytotoxic domain are linked to the molecular LM via a chemical bond, e.g. via an amide or peptide bond between an amino group of the LM and a carboxyl group, or its equivalent, of the TF antagonist and the cytotoxic domain, or vice versa. It is to be understood, that the LM may contain both covalent and non-covalent chemical bonds or mixtures thereof. The LM may comprise a plurality of carbon-carbon σ bonds having free rotation about their axes, so as to allow the TF antagonist and the cytotoxic domain to be separated by a distance suitable to both bind the TF site and elicit the effect of the cytotocix domain.

Suitable LMs, or backbones, comprise group(s) such as, but are not limited to, peptides; polynucleotides; sacharides including monosaccharides, di- and oligosaccharides, cyclodextrins and dextran; polymers including polyethylene glycol, polypropylene glycol, polyvinyl alcohol, hydrocarbons, polyacrylates and amino-, hydroxy-, thio- or carboxy-functionalised silicones, other biocompatible material units; and combinations thereof. Such LM materials described above are widely commercially available or obtainable via synthetic organic methods commonly known to those skilled in the art. In a preferred embodiment of the invention, the LM functions to release the cytotoxic domain of the compound having the formula A—(LM)—C. In one embodiment, the LM functions to release the cytotoxic compound following transfer to a reducing environment, e.g., cytoplasm or lysosomes. In another embodiment of the invention, the LM functions to release the cytotoxic compound following hydrolysis by specific hydrolases either inside the cell or on the cell surface, e.g. lysosomal proteases, such as Cathepsin B. In one embodiment of the invention, the LM functions to release the cytotoxic compound following an exogenous stimuli, e.g., light or other electromagnetic field radiation or ultrasound, e.g. high intensity focused ultrasound (HIFU).

The LM may, for example, comprise the following structures: straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl. The LM may be straight chained or branched and may contain one or more double or triple bonds. The LM may contain one or more heteroatoms like N, O or S. It is to be understood, that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

By “combinations thereof” is meant that the LM can comprise more than one class of the groups described above, as well as being able to comprise more than one member within a class. Where the LM comprises more than one class of group, such LM is preferably obtained by joining different units via their functional groups. Methods for forming such bonds involve standard organic synthesis and are well known to those of ordinary skill in the art.

The LM can comprise functional groups, including, without limitation, hydroxy, oxo, amino, C₁₋₄ monoalkylamino, acylamino, sulfonyl, carboxy, carboxamido, halogeno, C₁₋₆ alkoxy, C₁₋₆ alkylthio, trifluoroalkoxy, alkoxycarbonyl, or haloalkyl groups. The LM can also comprise charged functional groups, such as, for example, ammonium groups or carboxylate groups.

The charged functional groups can provide TF antagonists with sufficient solubility in aqueous or physiological systems, provide reactive sites for ionic bonding with other species, and enhance their avidity to other members of the TF/FVIIa/FXa complex. It is within the purview of one of skill in the art to select a particular acid, and concentration thereof, to confer optimal solubility and avidity properties to the TF antagonists. Preferably, the total amount of charged functional groups are minimised so as to maximise the TF antagonists specificity for TF sites, but not so as to significantly decrease solubility.

The terms “C₁₋₅₀-alkyl” or “C₁₋₅₀-alkanediyl” as used herein, refer to a straight or branched, saturated or unsaturated hydrocarbon chain having from one to 50 carbon atoms.

The terms “C₂₋₅₀-alkenyl” or “C₂₋₅₀-alkenediyl” as used herein, refer to an unsaturated branched or straight hydrocarbon chain having from 2 to 50 carbon atoms and at least one double bond.

The terms “C₂₋₅₀-alkynyl” or “C₂₋₅₀-alkynediyl” as used herein, refer to an unsaturated branched or straight hydrocarbon chain having from 2 to 50 carbon atoms and at least one triple bond. The C₁₋₅₀-alkyl residues include aliphatic hydrocarbon residues, unsaturated aliphatic hydrocarbon residues, alicyclic hydrocarbon residues. Examples of a C₁₋₅₀-alkyl within this definition include, but are not limited to, decanyl, hexadecanyl, octadecanyl, nonadecanyl, icosanyl, docosanyl, tetracosanyl, triacontanyl, decanediyl, hexadecanediyl, octadecanediyl, nonadecanediyl, icosanediyl, docosanediyl, tetracosanediyl, triacontanediyl,

The term C₃₋₈-cycloalkyl means an alicyclic hydrocarbon residue including saturated alicyclic hydrocarbon residues having 3 to 8 carbon atoms such as cyclopropyl, cyclobutyl, cyclopentyl, cyclohexyl; and C₅₋₆ unsaturated alicyclic hydrocarbon residues having 5 to 6 carbon atoms such as 1-cyclopentenyl, 2-cyclopentenyl, 3-cyclopentenyl, 1-cyclohexenyl, 2-cyclohexenyl, 3-cyclohexenyl.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms e.g. methoxy, ethoxy, propoxy, isopropoxy, butoxy, pentoxy.

The term “C₁₋₆-alkylthio” as used herein, alone or in combination, refers to a straight or branched monovalent substituent comprising a C₁₋₆-alkyl group linked through an thioether sulfur atom having its free valence bond from the thioether sulfur and having 1 to 6 carbon atoms.

The terms “aryl” and “heteroaryl” as used herein refers to an aryl which can be optionally substituted or a heteroaryl which can be optionally substituted and includes phenyl, biphenyl, indene, fluorene, naphthyl (1-naphthyl, 2-naphthyl), anthracene (1-anthracenyl, 2-anthracenyl, 3-anthracenyl), thiophene (2-thienyl, 3-thienyl), furyl (2-furyl, 3-furyl), indolyl, oxadiazolyl, isoxazolyl, quinazolin, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3- pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydrobenzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl).

The invention also relates to partly or fully saturated analogues of the ring systems mentioned above.

The terms “C₁₋₄ monoalkylamino” and “C₁₋₄ dialkylamino” refer to an amino group having one or both of its hydrogens independently replaced by an alkyl group having 1 to 4 carbon atoms, alkyl being defined above, such as methylamino, dimethylamino, N-ethyl-N-methylamino, ethylamino, diethylamino, propylamino, dipropylamino, N-(n-butyl)-N-methylamino, n-butylamino, di(n-butyl)amino, sec-butylammino, t-butylamino, and the like.

The terms “acyl” or “carboxy” refer to a monovalent substituent comprising a C₁₋₆-alkyl group linked through a carbonyl group; such as e.g. acetyl, propionyl, butyryl, isobutyryl, pivaloyl, valeryl, and the like.

The term “acylamino” refers to the group C₁₋ₙC(═O)NH—

The term “carboxamido” refers to the group —C(═O)NHC₁₋ₙ

The term “trifluoroalkoxy” refers to an C₁₋₆ alkoxy group as defined above having three of its hydrogen atoms bonded to one or more of the carbon atoms replaced by fluor atoms, such as (CF₃)O—, (CF₃)CH₂O—.

The term “alkoxycarbonyl” refers to the group —C(═O)(R) where R is an C₁₋₆ alkoxy group as defined above. The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, secbutoxycarbonyl, tert-butoxycarbonyl, 3-methylbutoxycarbonyl, n-hexoxycarbonyl and the like.

The term “leaving group” as used herein includes, but is not limited to, halogen, sulfonate or an acyl group. Suitable leaving groups will be known to a person skilled in the art.

“Halogen” refers to fluorine, chlorine, bromine, and iodine. “Halo” refers to fluoro, chloro, bromo and iodo.

“Optional” or “optionally” means that the subsequently described event or circumstances may or may not occur, and that the description includes instances where said event or circumstance occur and instances in which is does not. For example, “aryl . . . optionally substituted” means that the aryl may or may not be substituted and that the description includes both unsubstituted aryls and aryls wherein there is substitution

In one embodiment of the invention, LM comprises a FVIIa inhibitor. It is to be understood, that the FVIIa inhibitor is used to conjugate the cytotoxic domain via LM comprising the FVIIa inhibitor into the active site of a FVIIa polypeptide.

The cytotoxic domain linker moiety conjugates C—(LM) comprising a FVIIa inhibitor to be used in the preparation of a TF antagonist may be prepared by the following methods. In the following methods the FVIIa inhibitor is designated the letter F. The cytotoxic domain C is designated the letter C. Linker part B refers to other linker part of the LM.

Method 1.

LM comprising FVIIa inhibitors is prepared by reacting F—B—X, in which X is a functional group capable of reacting with structures C—Y, in which Y is a functional group, by means of normal coupling reactions using coupling reagents known by the person skilled in the art.

Method 2.

LM comprising FVIIa inhibitors may be prepared by reaction between F—B—Z, in which Z is a leaving group and C—W in which W is a nucleofile. Examples of leaving groups are halogens, sulfonates, phosphonates. Examples of nucleofiles are hydroxy, amino, N-substituted amino, and carbanions.

Method 3.

LM comprising FVIIa inhibitors may be prepared by reaction between C—B—Z, in which Z is a leaving group, and F—W, in which W is a nucleofile. Examples of leaving groups are halogens, sulfonates, phosphonates. Examples of nucleofiles are hydroxy, amino, N-substituted amino, and carbanions.

Method 4.

The linker part B can be reacted with structures F and C connected to a solid phase surface using methods well known in the art.

Method 5.

The cytotoxic domain-linker moiety conjugates C—(LM) comprising a FVIIa inhibitor may be prepared by a sequence of reactions through which F or C firstly are reacted with the activated linker moiety forming F—B, respectively C—B moieties and subsequently the formed product is reacted with C, respectively F moiety. The actual bond formation taking place through reaction on functional groups or derivatives or leaving groups/nucleofiles as described under methods 1-3.

The reaction can be carried out in solution phase or on a solid phase support using procedures known by the person skilled in the art.

In the present specification, amino acids are represented using abbreviations, as indicated in table 1, approved by IUPAC-IUB Commission on Biochemical Nomenclature (CBN). Amino acid and the like having isomers represented by name or the following abbreviations are in natural L-form unless otherwise indicated. Further, the left and right ends of an amino acid sequence of a peptide are, respectively, the N- and C-termini unless otherwise specified.

TABLE 1
Abbreviations for amino acids:

| Amino acid | Tree-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The invention also relates to a method of preparing TF antagonists as mentioned above. The TF antagonist may be produced by recombinant DNA techniques. To this end, DNA sequences encoding human FVIIa may be isolated by preparing a genomic or cDNA library and screening for DNA sequences coding for all or part of the protein by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the DNA sequence encoding the protein is preferably of human origin, i.e. derived from a human genomic DNA or cDNA library.

The DNA sequences encoding the human FVIIa polypeptides may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The DNA sequences encoding the human FVIIa polypeptides are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the human FVIIa polypeptides is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the human FVIIa polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the human FVIIa polypeptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may also contain a set of RNA splice sites located downstream from the promoter and upstream from the insertion site for the FVIIa sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 Elb region, the human growth hormone gene terminator (DeNoto et al. Nuc. Acids Res. 9:3719-3730, 1981) or the polyadenylation signal from the human FVII gene or the bovine FVII gene. The expression vectors may also include a noncoding viral leader sequence, such as the adenovirus 2 tripartite leader, located between the promoter and the RNA splice sites; and enhancer sequences, such as the SV40 enhancer.

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp.125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

To direct the human FVIIa polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the human FVIIa polypeptides in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed human FVIIa polypeptides into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the human FVIIa polypeptides. The function of the leader peptide is to allow the expressed peptide to be directed from the endoplasmic reticulum to the Golgi apparatus and further to a secretory vesicle for secretion into the culture medium (i.e. exportation of the human FVIIa polypeptides across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral (α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the human FVIIa polypeptides, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1 982), 841-845.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the human FVIIa polypeptides of interest. Media generally include a carbon source, a nitrogen source, essential amino acids, essential sugars, vitamins, salts, phospholipids, protein and growth factors. For production of gamma-carboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the human FVIIa polypeptide of interest.

The host cell into which the DNA sequences encoding the human FVIIa polypeptides is introduced may be any cell, which is capable of producing the posttranslational modified human FVIIa polypeptides and includes yeast, fungi and higher eucaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk⁻ ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk⁻ ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. No. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the human FVIIa polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the human FVIIa polypeptide after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The human FVIIa polypeptide produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

For the preparation of recombinant human FVIIa polypeptides, a cloned wild-type FVIIa DNA sequence is used. This sequence may be modified to encode a desired FVIIa variant. The complete nucleotide and amino acid sequences for human FVIIa are known. See U.S. Pat. No. 4,784,950, which is incorporated herein by reference, where the cloning and expression of recombinant human FVIIa is described. The bovine FVIIa sequence is described in Takeya et al., J. Biol Chem, 263:14868-14872 (1988), which is incorporated by reference herein.

The amino acid sequence alterations may be accomplished by a variety of techniques. Modification of the DNA sequence may be by site-specific mutagenesis. Techniques for site-specific mutagenesis are well known in the art and are described by, for example, Zoller and Smith (DNA 3:479-488, 1984). Thus, using the nucleotide and amino acid sequences of FVII, one may introduce the alterations of choice.

DNA sequences for use within the present invention will typically encode a pre-pro peptide at the amino-terminus of the FVIIa protein to obtain proper post-translational processing (e.g. gamma-carboxylation of glutamic acid residues) and secretion from the host cell. The pre-pro peptide may be that of FVIIa or another vitamin K-dependent plasma protein, such as factor IX, factor X, prothrombin, protein C or protein S. As will be appreciated by those skilled in the art, additional modifications can be made in the amino acid sequence of FVIIa where those modifications do not significantly impair the ability of the protein to act as a coagulation factor. For example, FVIIa in the catalytic triad can also be modified in the activation cleavage site to inhibit the conversion of zymogen FVII into its activated two-chain form, as generally described in U.S. Pat. No. 5,288,629, incorporated herein by reference.

Within the present invention, transgenic animal technology may be employed to produce the human FVIIa polypeptide. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and well characterized biochemically. Furthermore, the major milk proteins are present in milk at high concentrations (typically from about 1 to 15 g/l). From a commercial point of view, it is clearly preferable to use as the host a species that has a large milk yield. While smaller animals such as mice and rats can be used (and are preferred at the proof of principle stage), within the present invention it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk. See WIPO Publication WO 88/00239 for a comparison of factors influencing the choice of host species. It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489, incorporated herein by reference), beta-lactoglobulin, alpha-lactalbumin, and whey acidic protein. The beta-lactoglobulin (BLG) promoter is preferred. In the case of the ovine beta-lactoglobulin gene, a region of at least the proximal 406 bp of 5′ flanking sequence of the gene will generally be used, although larger portions of the 5′ flanking sequence, up to about 5 kbp, are preferred, such as about 4.25 kbp DNA segment encompassing the 5′ flanking promoter and non-coding portion of the beta-lactoglobulin gene. See Whitelaw et al., Biochem J. 286: 31-39 (1992). Similar fragments of promoter DNA from other species are also suitable.

Other regions of the beta-lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836-840 (1988); Palmiter et al., Proc. Natl. Acad. Sci. USA 88: 478-482 (1991); Whitelaw et al., Transgenic Res. 1: 3-13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta-lactoglobulin gene, is preferred. One such region is a DNA segment which provides for intron splicing and RNA polyadenylation from the 3′ non-coding region of the ovine beta-lactoglobulin gene. When substituted for the natural 3′ non-coding sequences of a gene, this ovine beta-lactoglobulin segment can both enhance and stabilize expression levels of the protein or polypeptide of interest. Within other embodiments, the region surrounding the initiation ATG of the sequence encoding the human FVIIa polypeptide is replaced with corresponding sequences from a milk specific protein gene. Such replacement provides a putative tissue-specific initiation environment to enhance expression. It is convenient to replace the entire pre-pro sequence of the human FVIIa polypeptide and 5′ non-coding sequences with those of, for example, the BLG gene, although smaller regions may be replaced.

For expression of a human FVIIa polypeptide in transgenic animals, a DNA segment encoding the human FVIIa polypeptide is operably linked to additional DNA segments required for its expression to produce expression units. Such additional segments include the above-mentioned promoter, as well as sequences which provide for termination of transcription and polyadenylation of mRNA. The expression units will further include a DNA segment encoding a secretory signal sequence operably linked to the segment encoding the human FVIIa polypeptide. The secretory signal sequence may be a native secretory signal sequence of the human FVIIa polypeptide or may be that of another protein, such as a milk protein. See, for example, von Heinje, Nuc. Acids Res. 14: 4683-4690 (1986); and Meade et al., U.S. Pat. No. 4,873,316, which are incorporated herein by reference.

Construction of expression units for use in transgenic animals is conveniently carried out by inserting a sequence encoding the human FVIIa polypeptide into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of the human FVIIa polypeptide, thereby creating a gene fusion that includes the expression control sequences of the milk protein gene. In any event, cloning of the expression units in plasmids or other vectors facilitates the amplification of the human FVIIa polypeptide. Amplification is conveniently carried out in bacterial (e.g. E. coli) host cells, thus the vectors will typically include an origin of replication and a selectable marker functional in bacterial host cells.

The expression unit is then introduced into fertilized eggs (including early-stage embryos) of the chosen host species. Introduction of heterologous DNA can be accomplished by one of several routes, including microinjection (e.g. U.S. Pat. No. 4,873,191), retroviral infection (Jaenisch, Science 240: 1468-1474 (1988)) or site-directed integration using embryonic stem (ES) cells (reviewed by Bradley et al., Bio/Technology 10: 534-539 (1992)). The eggs are then implanted into the oviducts or uteri of pseudopregnant females and allowed to develop. Offspring carrying the introduced DNA in their germ line can pass the DNA on to their progeny in the normal, Mendelian fashion, allowing the development of transgenic herds.

General procedures for producing transgenic animals are known in the art. See, for example, Hogan et al., Manipulating the Mouse Embryo: A Laboratory Manual, Cold Spring Harbor Laboratory, 1986; Simons et al., Bio/Technology6: 179-183 (1988); Wall et al., Biol. Reprod. 32: 645-651 (1985); Buhler et al., Bio/Technology 8: 140-143 (1990); Ebert et al., Bio/Technology 9: 835-838 (1991); Krimpenfort et al., Bio/Technology 9: 844-847 (1991); Wall et al., J. Cell. Biochem. 49:113-120 (1992); U.S. Pat. Nos. 4,873,191 and 4,873,316; WIPO publications WO 88/00239, WO 90/05188, WO 92/11757; and GB 87/00458, which are incorporated herein by reference. Techniques for introducing foreign DNA sequences into mammals and their germ cells were originally developed in the mouse. See, e.g., Gordon et al., Proc. Natl. Acad. Sci. USA 77: 7380-7384 (1980); Gordon and Ruddle, Science214: 1244-1246 (1981); Palmiter and Brinster, Cell 41: 343-345 (1985); and Brinster et al., Proc. Natl. Acad. Sci. USA 82: 4438-4442 (1985). These techniques were subsequently adapted for use with larger animals, including livestock species (see e.g., WIPO publications WO 88/00239, WO 90/05188, and WO 92/11757; and Simons et al., Bio/Technology 6: 179-183 (1988). To summarize, in the most efficient route used to date in the generation of transgenic mice or livestock, several hundred linear molecules of the DNA of interest are injected into one of the pro-nuclei of a fertilized egg according to established techniques. Injection of DNA into the cytoplasm of a zygote can also be employed. Production in transgenic plants may also be employed. Expression may be generalized or directed to a particular organ, such as a tuber. See, Hiatt, Nature 344:469-479 (1990); Edelbaum et al., J. Interferon Res. 12:449-453 (1992); Sijmons et al., Bio/Technology 8:217-221 (1990); and European Patent Office Publication EP 255,378.

FVIIa produced according to the present invention may be purified by affinity chromatography on an anti-FVII antibody column. It is preferred that the immunoadsorption column comprise a high-specificity monoclonal antibody. The use of calcium-dependent monoclonal antibodies, as described by Wakabayashi et al., J. Biol Chem. 261:11097-11108, (1986) and Thim et al., Biochem. 27: 7785-7793, (1988), incorporated by reference herein, is particularly preferred. Additional purification may be achieved by conventional chemical purification means, such as high performance liquid chromatography. Other methods of purification, including barium citrate precipitation, are known in the art, and may be applied to the purification of the FVIIa described herein (see, generally, Scopes, R., Protein Purification, Springer-Verlag, N.Y., 1982). Substantially pure FVIIa of at least about 90 to 95% homogeneity is preferred, and 98 to 99% or more homogeneity most preferred, for pharmaceutical uses. Once purified, partially or to homogeneity as desired, the FVIIa may then be used therapeutically.

Conversion of single-chain FVII to active two-chain FVIIa may be achieved using factor XIIa as described by Hedner and Kisiel (1983, J. Clin. Invest. 71: 1836-1841), or with other proteases having trypsin-like specificity (Kisiel and Fujikawa, Behring Inst. Mitt. 73: 29-42, 1983). Alternatively FVII may be autoactivated by passing it through an ion-exchange chromatography column, such as mono Q.RTM. (Pharmacia Fire Chemicals) or the like (Bjoern et al., 1986, Research Disclosures 269:564-565). The FVIIa molecules of the present invention and pharmaceutical compositions thereof are particularly useful for administration to humans to treat a variety of conditions involving intravascular coagulation.

The compounds of the present invention may have one or more asymmetric centres and it is intended that stereoisomers (optical isomers), as separated, pure or partially purified stereoisomers or racemic mixtures thereof are included in the scope of the invention.

Within the present invention, the TF antagonist may be prepared in the form of pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Further examples of pharmaceutically acceptable inorganic or organic acid addition salts include the pharmaceutically acceptable salts listed in Journal of Pharmaceutical Science, 66, 2 (1977) which are known to the skilled artisan.

Also intended as pharmaceutically acceptable acid addition salts are the hydrates which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of this invention may form solvates with standard low molecular weight solvents using methods known to the skilled artisan.

The TF antagonists of the invention are useful for the preparation of a pharmaceutical composition for the treatment of or prophylaxis of thrombotic or coagulopathic related diseases or disorders including vascular diseases and inflammatory responses. Such diseases and responses include, but are not limited to deep venous thrombosis, arterial thrombosis, post surgical thrombosis, coronary artery bypass graft (CABG), percutaneous transdermal coronary angioplastry (PTCA), stroke, tumour metastasis, inflammation, septic chock, hypotension, ARDS, pulmonary embolism, disseminated intravascular coagulation (DIC), vascular restenosis, platelet deposition, myocardial infarction, angiogenesis, or the prophylactic treatment of mammals with atherosclerotic vessels at risk for thrombosis.

The TF antagonist may be administered in pharmaceutically acceptable acid addition salt form or, where appropriate, as a alkali metal or alkaline earth metal or lower alkylammonium salt. Such salt forms are believed to exhibit approximately the same order of activity as the free base forms.

Apart from the pharmaceutical use of the compounds, they may be useful in vitro tools for investigating the inhibition of FVIIa, FXa or TF/FVIIa/FXa activity.

Pharmaceutical Compositions

In another aspect, the present invention includes within its scope pharmaceutical compositions comprising (i) a TF antagonist, as an active ingredient, or a pharmaceutically acceptable salt thereof together with (ii) a pharmaceutically acceptable carrier or diluent.

Optionally, the pharmaceutical composition of the invention may comprise a TF antagonist in combination with one or more other compounds exhibiting anticoagulant activity, e.g., platelet aggregation inhibitor.

The compounds of the invention may be formulated into pharmaceutical composition comprising the compounds and a pharmaceutically acceptable carrier or diluent. Such carriers include water, physiological saline, ethanol, polyols, e.g., glycerol or propylene glycol, or vegetable oils. As used herein, “pharmaceutically acceptable carriers” also encompasses any and all solvents, dispersion media, coatings, antifungal agents, preservatives, isotonic agents and the like. Except insofar as any conventional medium is incompatible with the active ingredient and its intended use, its use in the compositions of the present invention is contemplated.

The compositions may be prepared by conventional techniques and appear in conventional forms, for example, capsules, tablets, solutions or suspensions. The pharmaceutical carrier employed may be a conventional solid or liquid carrier. Examples of solid carriers are lactose, terra alba, sucrose, talc, gelatine, agar, pectin, acacia, magnesium stearate and stearic acid. Examples of liquid carriers are syrup, peanut oil, olive oil and water. Similarly, the carrier or diluent may include any time delay material known to the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art.

The pharmaceutical compositions can be sterilised and mixed, if desired, with auxiliary agents, emulsifiers, salt for influencing osmotic pressure, buffers and/or colouring substances and the like, which do not deleteriously react with the active compounds.

The route of administration may be any route, which effectively transports the active compound to the appropriate or desired site of action, such as oral or parenteral, e.g., rectal, transdermal, subcutaneous, intranasal, intramuscular, topical, intravenous, intraurethral, ophthalmic solution or an ointment, the oral route being preferred.

If a solid carrier for oral administration is used, the preparation can be tabletted, placed in a hard gelatine capsule in powder or pellet form or it can be in the form of a troche or lozenge. The amount of solid carrier may vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain a compound of formula (I) dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet, which may be prepared by conventional tabletting techniques, contains

| Core: | |
|---|---|
| Active compound (as free compound | 10 mg |
| or salt thereof) | |
| Colloidal silicon dioxide (Areosil ®) | 1.5 mg |
| Cellulose, microcryst. (Avicel ®) | 70 mg |
| Modified cellulose gum (Ac-Di-Sol ®) | 7.5 mg |

Magnesium Stearate

| Coating: | |
|---|---|
| HPMC | approx. 9 mg |
| *Mywacett ® 9-40 T | approx. 0.9 mg |

*Acylated monoglyceride used as plasticizer for film coating.

The compounds of the invention may be administered to a mammal, especially a human in need of such treatment, prevention, elimination, alleviation or amelioration of various thrombolytic or coagulophatic diseases or disorders as mentioned above. Such mammals also include animals, both domestic animals, e.g. household pets, and non-domestic animals such as wildlife.

Usually, dosage forms suitable for oral, nasal, pulmonal or transdermal administration comprise from about 0.001 mg to about 100 mg, preferably from about 0.01 mg to about 50 mg of the compounds of formula I admixed with a pharmaceutically acceptable carrier or diluent.

The compounds may be administered concurrently, simultaneously, or together with a pharmaceutically acceptable carrier or diluent, whether by oral, rectal, or parenteral (including subcutaneous) route. The compounds are often, and preferably, in the form of an alkali metal or earth alkali metal salt thereof.

Suitable dosage ranges varies as indicated above depending upon the exact mode of administration, form in which administered, the indication towards which the administration is directed, the subject involved and the body weight of the subject involved, and the preference and experience of the physician or veterinarian in charge.

The compounds of the present invention have interesting pharmacological properties. For example, the compounds of this invention can be used to modulate and normalise an impaired haemostatic balance in mammals caused by deficiency or malfunction of blood clotting factors or their inhibitors. The FVIIa and in particular the TF/FVIIa activity plays an important role in the control of the coagulation cascade, and modulators of this key regulatory activity such as the present invention can be used in the treatment of or prophylaxis of thrombotic or coagulopathic related diseases or disorders including vascular diseases and inflammatory responses. The pharmaceutical composition of the invention may thus be useful for modulating and normalising an impaired haemostatic balance in a mammal. In particular, the pharmaceutical composition may be useful for the treatment of or prophylaxis of thrombotic or coagulopathic related diseases or disorders including vascular diseases and inflammatory responses.

“Modulating and normalising an impaired haemostatic balance” means achieving an effect on the coagulation system measurable in vitro assays and/or animal models which diminishes the risk for thrombosis or bleedings.

More particularly, the pharmaceutical composition may be useful as an inhibitor of blood coagulation in a mammal, as an inhibitor of clotting activity in a mammal, as an inhibitor of deposition of fibrin in a mammal, as an inhibitor of platelet deposition in a mammal, in the treatment of mammals suffering from deep venous thrombosis, arterial thrombosis, post surgical thrombosis, coronary artery bypass graft (CABG), percutaneous transdermal coronary angioplastry (PTCA), stroke, tumour metastasis, inflammation, septic chock, hypotension, ARDS, pulmonary embolism, disseminated intravascular coagulation (DIC), vascular restenosis, platelet deposition, myocardial infarction, angiogenesis, or the prophylactic treatment of mammals with atherosclerotic vessels at risk for thrombosis. The compositions of the invention may also be used as an adjunct in thrombolytic therapy.

Furthermore, the invention relates to a method for inhibiting the TF initiation activity in a mammal which method comprises administering an effective amount of at least one compound of the present invention, in combination with a pharmaceutical acceptable excipient and/or carrier to the mammal in need of such a treatment.

Assays

Inhibition of FVIIa/Phospholipids-Embedded TF-Catalyzed Activation of FX by TF Antagonists FXa Generation Assay (Assay 1):

In the following example all concentrations are final. Lipidated TF (10 pM), FVIIa (100 pM) and TF antagonist or FFR-rFVIIa (0-50 nM) in HBS/BSA (50 mM hepes, pH 7.4, 150 mM NaCl, 5 mM CaCl₂, 1 mg/ml BSA) are incubated 60 min at room temperature before FX (50 nM) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC₅₀ values for TF antagonist inhibition of FVIIa/lipidated TF-mediated activation of FX may be calculated. The IC₅₀ value for FFR-rFVIIa is 51 +/−26 pM in this assay.

Inhibition of FVIIa/Cell Surface TF-Catalyzed Activation of FX by TF Antagonists (Assay 2):

In the following example all concentrations are final. Monolayers of human lung fibroblasts WI-38 (ATTC No. CCL-75) or human bladder carcinoma cell line J82 (ATTC No. HTB-1) or human keratinocyte cell line CCD 1102Ker Tr (ATCC no. CRL-2310) constitutively expressing TF are employed as TF source in FVIIa/TF catalyzed activation of FX. Confluent cell monolayers in a 96-well plate are washed one time in buffer A (10 mM Hepes, pH 7.45, 150 mM NaCl, 4 mM KCl, and 11 mM glucose) and one time in buffer B (buffer A supplemented with with 1 mg/ml BSA and 5 mM Ca²⁺). FVIIa (1 nM), FX (135 nM) and varying concentrations of TF antagonist or FFR-rFVIIa in buffer B are simultaneously added to the cells. FXa formation is allowed for 15 min at 37° C. 50-μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of colour development are converted to FXa concentrations using a FXa standard curve. The IC50 value for FFR-rFVIIa is 1.5 nM in this assay.

Inhibition of ¹²⁵I-FVIIa binding to Cell Surface TF by TF Antagonists (Assay 3):

In the following example all concentrations are final. Binding studies are employed using the human bladder carcinoma cell line J82 (ATTC No. HTB-1) or the human keratinocyte cell line (CCD1102KerTr ATCC No CRL-2310) or NHEK P166 (Clonetics No. CC-2507) all constitutively expressing TF. Confluent monolayers in 24-well tissue culture plates are washed once with buffer A (10 mM Hepes, pH 7.45, 150 mM NaCl, 4 mM KCl, and 11 mM glucose) supplemented with 5 mM EDTA and then once with buffer A and once with buffer B (buffer A supplemented with with 1 mg/ml BSA and 5 mM Ca²⁺). The monolayers are preincubated 2 min with 100 μl cold buffer B. Varying concentrations of Mabs (or FFR-FVIIa) and radiolabelled FVIIa (0.5 nM ¹²⁵I-FVIIa) are simultaneously added to the cells (final volume 200 μl). The plates are incubated for 2 hours at 4° C. At the end of the incubation, the unbound material is removed, the cells are washed 4 times with ice-cold buffer B and lysed with 300 μl lysis buffer (200 mM NaOH, 1% SDS and 10 mM EDTA). Radioactivity is measured in a gamma counter (Cobra, Packard Instruments). The binding data are analyzed and curve fitted using GraFit4 (Erithacus Software, Ltd., (U.K.). The IC₅₀ value for FFR-rFVIIa is 4 nM in this assay.

Biosensor Assay (Assay 4):

TF antagonists are tested on the Biacore instrument by passing a standard solution of the TF antagonist over a chip with immobilized TF. This is followed by different concentrations of sTF in 10 mM hepes pH 7.4 containing 150 mM NaCl, 10 mM CaCl₂ and 0.0003% polysorbate 20. Kd's are calculated from the sensorgrams using the integrated Biacore evaluation software.

Inhibition of FVIIa/TF-Induced p44/42 MAPK Activation by TF Antagonists with Effector Domain (Assay 5):

The amount of phosphorylated p44/42 MAPK and/or Akt, and/or p90RSK is determined by quantitative detection of chemiluminescence (Fujifilm LAS-1000) from western blot analysis. Cells expressing human TF, e.g. CCD1102KerTr, NHEK P166, human glioblastoma cell line U87, or human breast cancer cell line MDA-MB231, are cultured in medium with 0-0.1% FCS for 24 or 48 hours prior to the experiment to make cells quiescent. At the day of the experiment the cells must be 70-80% confluent. The experiment is performed by preincubating the cells with excess TF antagonist or FFR-rFVIIa in medium without serum for 30 min at 37° C. before addition of 10-100 nM FVIIa and incubating for 10 min. As a positive control of cell signaling, cells are treated with 10% FCS for 10 minutes. Cells are washed 2 times in ice-cold PBS before cells are lysed in lysis buffer (20 mM Tris, 0.1% Triton X-100, 1 mM EDTA, 1 mM EGTA, 50 mM sodium-fluoride, 10 mM sodium 0-glycerophosphate, 5 mM sodium pyrophosphate, 150 mM NaCl, pH 7.5 containing 0.1 mM 4-(2-aminoethyl)benzene-sulfonyl fluoride (AEBSF) and 1 mM benzamidine. Added just before use: 1 mM sodium orthovanadate, 5 μg/ml leupeptin, 10 μg/ml aprotinin). Lysates were mixed with SDS-sample buffer and loaded on a SDS-polyacrylamide gel. A standard biotinylated protein marker is loaded on each gel. Proteins separated on the SDS-polyacrylamide gel were transferred to nitrocellulose by electroblotting, and the kinases p44/42 MAPK, Akt and p90RSK were visualized by immunoblotting with phosphospecific antibodies, and chemiluminiscence is quanitiated by Fujifilm LAS1000.

The present invention is further illustrated by the following examples.

The present invention is not to be limited in scope by the specific embodiments disclosed in the examples which are intended as illustrations of a number of aspects of the invention and any embodiments which are functionally equivalent are within the scope of this invention. Those skilled in the art will know, or be able to ascertain using no more than routine experimentation, many equivalents to the specific embodiments of the invention described herein. These and all other equivalents are intended to be encompassed by the following claims.

### EXAMPLES

### Example 1

Generic Construct of Cytotoxic Domain Linker Moiety Conjugates C—(LM) for Intracellular Release of Cytotoxic Domain from TF Antagonist by Reduction:

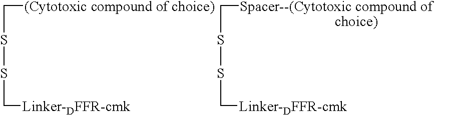

In the schematic illustration, the cytotoxic compound of choice may be any cytotoxic compound or domain which contains a group linkable to directly or via a spacer to a free thiol. The concept is illustrated with the FVIIa inhibitor D-Phe-Phe-Arg chloromethyl ketone (_{D}FFR-cmk) but may be expanded to all other FVIIa inhibitors and thiol containing compounds that may be coupled to the FVIIa inhibitor. The “linker” and “spacer” refers to other parts of the LM, which separates the cytotoxic domain from the FVIIa polypeptide. —S—S— refers to disulhide bond, which may be broken by reduction. It is to be understood that the cytotoxic domain linker moiety conjugates C—(LM) comprising the FVIIa inhibitor is reacted with FVIIa polypeptide to get the TF antagonist of the invention. The cytotoxic domain will be released in a functional form following transfer or internalization to a reducing environment, e.g., cytoplasm or lysosomes.

### Example 2

Specific Construct of Cytotoxic Domain Linker Moiety Conjugates C—(LM) for Intracellular Release of Cytotoxic (KLAKLAK)₂ Peptide from TF Antagonist by Reduction:

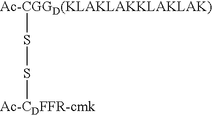

### Example 3

Generic Construct of Cytotoxic Domain Linker Moiety Conjugates C—(LM) for Release of Cytotoxic Domain from TF Antagonist by Hydrolysis:

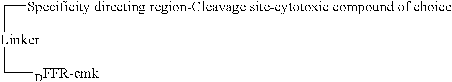

In the schematic illustration, the cytotoxic compound of choice may be any cytotoxic compound or domain which contains a group linkable to a specificity directing region (SDR) including a cleavage site via a ester or peptide bond. The properties of the SDR would depend on the hydrolase targeted, i.e. a lipase or a protease. If the target is a protease the SDR would be a peptide sequence known to the person skilled in the art. The concept is illustrated with the FVIIa inhibitor D-Phe-Phe-Arg chloromethyl ketone (_{D}FFR-cmk) but may be expanded to all other FVIIa inhibitors. The “linker” refers to other parts of the LM, which separates the cytotoxic domain from the FVIIa polypeptide. It is to be understood that the cytotoxic domain linker moiety conjugates C—(LM) comprising the FVIIa inhibitor is reacted with FVIIa polypeptide to get the TF antagonist of the invention. The cytotoxic domain will be released in a functional form following hydrolysis by cell specific or environment specific, e.g. intracellular protease or lipase.

### Example 4

Specific Construct of Cytotoxic Domain Linker Moiety Conjugates C—(LM) for Intracellular Release of Cytotoxic Domain from TF Antagonist by Hydrolysis with Cathepsin B:

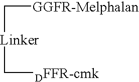

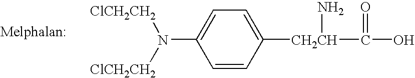

In the schematic illustration, melphalan conjugated via the amino acid sequence GGFR and a further optional linker part to the FVIIa inhibitor D-Phe-Phe-Arg chloromethyl ketone (_{D}FFR-cmk). The concept may be expanded to all other FVIIa inhibitors. The “linker” refers to other parts of the LM, which separates the cytotoxic domain from the FVII a polypeptide. It is to be understood that the cytotoxic domain linker moiety conjugates C—(LM) comprising the FVIIa inhibitor is reacted with FVIIa polypeptide to get the TF antagonist of the invention. The mephalan domain will be released in a functional form following hydrolysis by cathepsin B.

### Example 5

Generic Construct of Radionuclide Linker Moiety Conjugates C—(LM) for Delivery of TF Antagonist Containing Radionuclides:

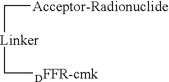

In the schematic illustration, the radionuclide of choice is conjugated to the FVIIa inhibitor D-Phe-Phe-Arg chloromethyl ketone (_{D}FFR-cmk). This may be expanded to all other FVIIa inhibitors. The “linker” refers to other parts of the LM, which separates the cytotoxic domain from the FVIIa polypeptide. It is to be understood that the radionuclide linker moiety conjugates C—(LM) comprising the FVIIa inhibitor is reacted with FVIIa polypeptide to get the TF antagonist of the invention. The radionuclide is delivered to or into the TF presenting cell.

The acceptor could be any group to which can be coupled a radionuclide using conventional chemistry, e.g. Bolton-Hunter.

### Example 6

Specific Construct of Radionuclide Linker Moiety Conjugates C—(LM) for Delivery of TF Antagonist Containing Radionuclides:

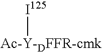

In the schematic illustration, the radionuclide I¹²⁵ is conjugated to the FVIIa inhibitor D-Phe-Phe-Arg chloromethyl ketone (_{D}FFR-cmk). This may be expanded to all other FVIIa inhibitors. It is to be understood that the radionuclide linker moiety conjugates C—(LM) comprising the FVII a inhibitor is reacted with FVII a polypeptide to get the TF antagonist of the invention. The radionuclide is delivered to or into the TF presenting cell. The acceptor could be any group to which can be coupled a radionuclide using conventional chemistry, e.g. Bolton-Hunter.

### Example 7

The following provide examples for the preparation of cytotoxic domain linker moiety conjugates C—(LM), wherein the cytotoxic domain is covalently conjugated to _{D}FFR-cmk (fFR-cmk) It is to be understood that the cytotoxic domain linker moiety conjugates C—(LM) comprising the FVIIa inhibitor is reacted with FVII a polypeptide to get the TF antagonist of the invention.

HPLC-MS Method

The following instrumentation is used:

Hewlett Packard series 1100 G1312A Bin Pump

Hewlett Packard series 1100 Column compartment

Hewlett Packard series 1100 G1315A DAD diode array detector

Hewlett Packard series 1100 MSD

Sedere 75 Evaporative Light Scattering detector

The instrument is controlled by HP Chemstation software.

The HPLC pump is connected to two eluent reservoirs containing:

| | |
|---|---|
| A | 0.01% TFA in water |
| B | 0.01% TFA in acetonitrile |

The analysis is performed at 40° C. by injecting an appropriate volume of the sample (preferably 1 μl) onto the column which is eluted with a gradient of acetonitrile.

The HPLC conditions, detector settings and mass spectrometer settings used are given in the following table.

| | |
|---|---|
| Column | Waters Xterra MS C-18 X 3 mm id 5 μm |
| Gradient | 5%-100% acetonitrile linear during 7.5 min at 1.5 |
| | ml/min |
| Detection | 210 nm (analogue output from DAD) |
| | ELS (analogue output from ELS) |
| MS | ionisation mode API-ES |
| | Scan 100-1000 amu step 0.1 amu |

After the DAD the flow is divided yielding approximately 1 ml/min to the ELS and 0.5 ml/min to the MS.

### Example 8

{4,7-Biscarboxymethyl-10-[(1-{1-[1-(2-chloroacetyl)-4-guanidinobutylcarbamoyl]-2-phenylethylcarbamoyl}-2-phenylethylcarbamoyl)methyl]-1,4,7,10-tetraazacyclododec-1-yl}acetic Acid (DOTA-fFRcmk).

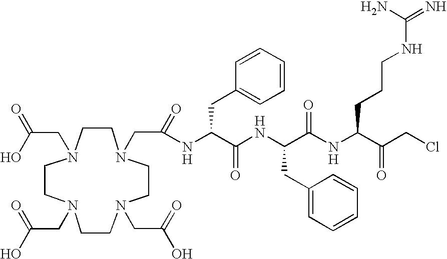

The trifluoroacetate salt of 2-amino-N-{1-[1-(2-chloroacetyl)-4-guanidinobutylcarbamoyl]-2-phenylethyl}-3-phenylpropionamide (fFRcmk.2 TFA) (40 mg, 0.055 mmol) and the hexafluorophosphate salt of [4,7-biscarboxymethyl-10-(2,5-dioxopyrrolidin-1-yloxycarbonylmethyl)-1,4,7,10-tetraazacyclododec-1-yl]acetic acid (DOTA-NHS ester. PF₆) (160 mg, 0.247 mmol) were dissolved in degassed N,N-dimethylformamide (0.64 mL). Sodium hydrogencarbonate (161 mg, 1.92 mmol) was added, and the suspension was stirred vigorously for 5 h at room temperature. A solution of trifluoroacetic acid (0.5 mL) in dichloromethane (5 mL) was added, and the resulting mixture was concentrated. Purification by flash chromatography on reversed-phase SiO₂ (C-18) yielded the title compound.

HPLC-MS: m/z: 887 (M+H); Rₜ=2.15 min.

### Example 9

2-(2-Acetylamino-3-{4-[bis(2-chloroethyl)amino]phenyl}propionylamino)-N-{1-[1-(2-chloroacetyl)-4-guanidinobutylcarbamoyl]-2-phenylethyl}-3-phenylpropionamide.

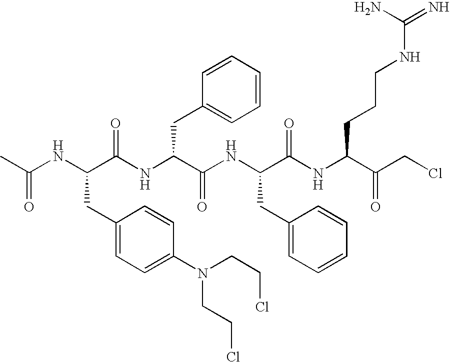

2-Amino-3-{4-[bis(2-chloroethyl)amino]phenyl}propionic acid (melphalan) (200 mg, 0.655 mmol) and sodium hydrogencarbonate (220 mg, 2.62 mmol) were suspended in 1,4-dioxane/water (1:1) (1 mL). Acetic anhydride (74 μL, 80 mg, 0.78 mmol) was added, and the reaction mixture was stirred vigorously at room temperature for 30 minutes. The resulting mixture was acidified to pH 5, by the addition of 1 N hydrochloric acid (aq.), and extracted with ethyl acetate (3×2 mL). The combined organic phase was dried with magnesium sulfate (1 h), filtered and concentrated to furnish 2-acetylamino-3-{4-[bis(2-chloroethyl)amino]phenyl}propionic acid (N-Ac-melphalan) (56 mg). No further purification was necessary.

¹H-NMR (DMSO-d₆): δ12.48 (1H, bs), 8.03 (1H, bd), 7.06 (2H, d), 6.64 (2H, d), 4.27 (1H, m), 3.69 (8H, bs), 2.91 (1 H, dd), 2.72 (1H, dd), 1.78 (3H, s). HPLC-MS: m/z: 347 (M+H); Rₜ=3.24 min.

The trifluoroacetate salt of 2-amino-N-{1-[1-(2-chloroacetyl)-4-guanidinobutylcarbamoyl]-2-phenylethyl}-3-phenylpropionamide (fFRcmk.2 TFA) (25 mg, 0.034 mmol) and 2-acetylamino-3-{4-[bis(2-chloroethyl)amino]phenyl}propionic acid (N-Ac-melphalan) (13 mg, 0.037 mmol) were dissolved in N,N-dimethylformamide (0.34 mL). 1-Hydroxybenzotriazol (HOBt) (4.6 mg, 0.034 mmol), sodium hydrogencarbonate (17 mg, 0.20 mmol) and 1-ethyl-3-(3-dimethylaminopropyl)carbodiimid hydrochloride (EDCI) (13 mg, 0.068 mmol) were added consecutively. The reaction mixture was stirred vigorously at room temperature for 1 hour. Trifluoroacetic acid (0.2 mL) was added, and the resulting mixture was concentrated. The crude product was purified by flash chromatography on SiO₂, eluting with dichloromethane/methanol (95:5), to yield the title compound (22 mg).

¹H-NMR (DMSO-d₆): δ8.61 (1H, d), 8.54 (1H, d), 8.26 (1H, d), 7.93 (1H, d), 7.44 (1H, t), 7.33-6.95 (10H, m), 6.90 (2H, d), 6.56 (2H, d), 4.55 (2H, m), 4.45 (1H, d), 4.36 (1H, d), 4.33 (2H, m), 3.67 (8H, bs), 3.09 (4H, m), 2.79 (3H, m), 2.59 (1H, m), 1.80 (1H, m), 1.70 (3H, s), 1.53 (1H, m), 1.43 (2H, m). HPLC-MS: m/z: 829 (M+H); Rₜ=3.64 min.

All patents, patent applications, and literature references referred to herein are hereby incorporated by reference in their entirety.

1. A compound having the formula A-(LM)-C, wherein A comprises a TF antagonist; LM comprises an optional linker moiety; and C comprises a cytotoxic domain.

2. The compound according to claim 1, wherein the TF antagonist is an inactive FVIIa polypeptide.

3. The compound according to claim 2, wherein the inactive FVIIa polypeptide is native human FVIIa or a fragment thereof catalytically inactivated in the active site.

4. The compound according to claim 3, wherein the inactive FVIIa polypeptide is native human FVIIa catalytically inactivated in the active site.

5. The compound according to claim 2, wherein C or (LM)-C is conjugated to the active site of the FVIIa polypeptide.

6. The compound according claim 2, wherein the FVIIa polypeptide is catalytically inactivated in the active site with a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone, and Dansyl-D-Glu-Gly-Arg chloromethylketone.

7. The compound according to claim 2, wherein LM comprises a chloromethyl ketone inhibitor independently selected from the group consisting of Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone, and Dansyl-D-Glu-Gly-Arg chloromethylketone, wherein the inactive FVIIa polypeptide is catalytically inactivated in the active site with said chloromethyl ketone inhibitor.

8. The compound according to claim 1, wherein the TF antagonist is an antibody against TF.

9. The compound according to claim 8, wherein the antibody is a human monoclonal antibody against human TF.

10. The compound according to claim 1, wherein C is selected from the group consisting of: protein ionophores, cytostatic agents, chemotherapeutic compounds, compounds which induce apoptosis, compounds containing radionuclides, and antisense nucleotide molecules independent selected from the group consisting of PNAs, DNAs, RNAs and LNAs.

11. The compound according to claim 1, wherein C comprises a cytotoxic protein or peptide.

12. The compound according to claim 11, wherein C comprises the amino acid sequence (KLAKLAK)ₙ, wherein n is selected from the group consisting of 1, 2, 3, 4, 5, 6, 7, and 8.

13. The compound according to claim 12, wherein C has the amino acid sequence (KLAKLAK)₂

14. The compound according to claim 1, wherein C or (LM)-C is conjugated to an oligosaccharide side chain present on A.

15. The compound according to claim 1, wherein C or (LM)-C is conjugated to a free sulfhydryl group present on A.

16. The compound according to claim 1, wherein the compound comprises more than one binding site for TF.

17. The compound according to claim 1, wherein LM comprises an amino acid sequence.

18. The compound according to claim 17, wherein LM comprises the amino acid sequence Gly-Gly.

19. The compound according to claim 1, wherein LM comprises a molecule selected from the group consisting of: straight or branched C₁₋₅₀-alkyl, straight or branched C₂₋₅₀-alkenyl, straight or branched C₂₋₅₀-alkynyl, a 1 to 50-membered straight or branched chain comprising carbon and at least one N, O or S atom in the chain, C₃₋₈cycloalkyl, a 3 to 8-membered cyclic ring comprising carbon and at least one N, O or S atom in the ring, aryl, heteroaryl, amino acid, the structures optionally substituted with one or more of the following groups: H, hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogeno, C₁₋₆ alkoxy, C₁₋₆ alkylthio, trifluoroalkoxy, alkoxycarbonyl, haloalkyl.

20. The compound according to claim 1, wherein LM comprises a chemical bond that can broken by chemical reduction.

21. The compound according to claim 20, wherein LM comprises a disulfide bond.

22. The compound according to claim 21, wherein the disulfide bond is between two cysteines.

23. The compound according to claim 1, wherein LM comprises a cleavage site for enzymatic hydrolysis by a protease.

24. The compound according to claim 23, wherein the protease is selected from the group consisting of cathepsin B, cathepsin D, cathepsin E, cathepsin G, cathepsin H, cathepsin L, cathepsin N, cathepsin S, cathepsin T, cathepsin K, and legumain.

25. The compound according to claim 24, wherein the protease is cathepsin B.

26. The compound according to claim 23, wherein LM comprises the amino acid sequence Phe-Arg.

27. A pharmaceutical composition comprising (i) an amount of the compound having the formula A-(LM)-C, wherein A comprises a TF antagonist; LM comprises an optional linker moiety; and C comprises a cytotoxic domain; and (ii) a pharmaceutically acceptable carrier or excipient.

28. A compound for use as a medicament having the formula A-(LM)-C, wherein A comprises a TF antagonist; LM comprises an optional linker moiety; and C comprises a cytotoxic domain; and wherein said compound binds to TF and inhibits TF function.

29. A method for preventing or treating disease or disorder associated with pathophysiological TF function, said method comprising contacting a TF presenting cell with a compound having the formula A-(LM)-C, wherein A is a TF antagonist; LM is an optional linker moiety; C comprises a cytotoxic domain.

30. The method according to claim 29, wherein the disease or disorder associated with pathophysiological TF function is selected from the group consisting of: deep venous thrombosis, arterial thrombosis, post surgical thrombosis, coronary artery bypass graft (CABG), percutaneous transdermal coronary angioplastry (PTCA), stroke, cancer, tumour metastasis, angiogenesis, ischemia/reperfusion, rheumatoid arthritis, thrombolysis, arteriosclerosis and restenosis following angioplastry, acute and chronic indications such as inflammation, septic chock, septicemia, hypotension, adult respiratory distress syndrome (ARDS), disseminated intravascular coagulopathy (DIC), pulmonary embolism, platelet deposition, myocardial infarction, and the prophylactic treatment of mammals with atherosclerotic vessels at risk for thrombosis.

31. A method for preventing or treating disease or disorder associated with pathophysiological TF function in a mammal in need of such treatment, said method comprising administering to said mammal a therapeutically effective amount of at least one compound as defined in claim 1 in combination with a pharmaceutical acceptable excipient and/or carrier.
